(19) United States
(12) Patent Application Publication
(10) Pub. No.: US 2002/0198600 A1
(43) Pub. Date: Dec. 26, 2002

(54) Stainless steel bone-shaped prosthesis for animals

(76) Inventor: Charles Kuntz, Springfield, VA (US)

Correspondence Address: Richard C. Litman, LITMAN LAW OFFICES, LTD., P.O. Box 15035, Arlington, VA, 22215, US

(21) Appl. No.: 10/161,628

(22) Filed: Jun. 5, 2002

Related U.S. Application Data
(60) Provisional application No. 60/300,437, filed on Jun. 26, 2001.

(51) Int. Cl. A61F 2/28

(52) U.S. Cl. 623/23.53; 606/71

(57) ABSTRACT

An artificial bone prosthesis for use in animals which replaces the portion of a bone lost as a result of disease, damage, or surgical removal. Because the life span of animals recovering from bone cancer and other bone diseases is very short, the bone prostheses used in bone replacement surgery in these animals do not have to allow for tissue or bone ingrowth, unlike similar prostheses used in humans. As such, the prosthesis of the present invention has a simple, unique shape that can be made of a conventional artificial material such as stainless steel. This makes the prosthesis relatively inexpensive and eliminates the problems arising in conventional natural bone transplants such as tissue rejection and bacterial infection.

### CROSS-REFERENCE TO RELATED APPLICATION

This application claims the benefit of U.S. Provisional Patent Application Serial No. 60/300,437, filed Jun. 26, 2001.

### BACKGROUND OF THE INVENTION

1. Field of the Invention

The present invention relates to artificial bone prostheses. More particularly, the present invention relates to artificial bone prostheses useful for animals.

2. DESCRIPTION OF RELATED ART

The present invention is an artificial bone prosthesis for use in animals which provides a number of benefits over traditional bone prostheses. The two primary therapies available to veterinarians and pet owners deciding on bone reconstruction surgery, namely natural bone transplants or artificial human-style prostheses, present a number of problems that make either of them unsatisfactory for use in bone reconstruction surgery in animals.

Veterinary medicine in general, and veterinary surgery in particular, has adopted a number of techniques and procedures for treating severe conditions in animals from similar treatments used to remedy similar problems in people. This is possible because the physiology of many common household pets, such as dogs and cats, is similar to that of humans. As such, these animals suffer from the same or similar ailments as humans, which in most cases can be remedied using treatments similar to those used to treat people. This allows veterinary medicine to benefit from the progress of human medicine. Newly discovered treatments for cancer in humans, for example, can be immediately applied to similar species that suffer from the same cancer.

The only drawback to this traditional approach is that many of the more sophisticated and life saving procedures to treat humans are prohibitively expensive when applied to animals. The treatment of bone cancer is a particularly revealing example of this problem.

In humans, bone cancer can be treated by a combination of drug therapies and surgery, which if successful can prolong the life of the patient for decades. In particular, the surgery used to treat bone cancer accomplishes a very specific purpose, the extraction of the cancerous tissue. Surgeons remove the portion of the bone afflicted with cancer in order to reduce the risk that the cancer will spread and to reduce pain and improve function. But while the bones are removed, the surrounding bones and muscle tissue are not, requiring an implant or prosthesis of some kind to provide structural support to the limb by replacing the missing portion of bone.

In the past, the implanted portion of bone was obtained from a similar segment of bone from a cadaver, organ donors, or bone bank. But despite being cut, cleaned, and prepared before implantation, many patients suffered from rejection of the new bone tissue or from bacterial or viral infection from the transplant. The solution to these problems was to use an artificial prosthesis rather than a natural one.

Conventional artificial prostheses for humans must have a number of characteristics. Because successfully treated bone cancer patients may survive for many years, implants subjected to repeated load cycling will eventually fail. So the most important feature of an artificial prosthesis is that it must in some way accommodate this new growth of the surrounding bone. Conventional prostheses for humans accomplish this function in a number of ways.

First, the prostheses commonly have a portion that is drilled into a portion or the center of the surrounding bone. Secondly, many prostheses have specially designed surface textures to which new bone tissue can attach. Finally, the prostheses have a shape and density that match as closely as possible the bone that was removed. This way, the prosthesis will feel more comfortable to the patient as they grow older.

Because of their complex design, these prostheses can be very expensive. They are often custom built to fit the specific patient that will receive it and they are made out of unique alloys that require special handling and preparation. In addition, because the prosthesis is attached directly to surrounding bones, the prosthesis surgery itself can be very long and complex. Typically though, people undergoing this procedure are covered by insurance. In addition, their families are usually willing to make any sacrifice to help them to obtain this procedure. This is not the case when pet owners are faced with the daunting cost of this procedure.

While this approach is very successful in restoring the longevity and quality of life in human patients, the situation is more complicated with animal patients. First, the life span for animals recovering from bone cancer is often only a few years. Second, the high cost of using artificial prostheses in animals may be more than pet owners are willing or capable of spending to prolong the life of their pet for those few short years. Because of this, many pet owners prefer, and many veterinary surgeons provide, conventional, amputation or limb salvage using natural bone implants as an alternative for synthetic bone reconstruction surgery.

But, the use of natural bones transplants in animals requiring bone reconstruction surgery poses all of the problems that arose when using natural bone transplants in humans, but also presents new problems that are unique to animals as well.

Specifically, the natural bone replacement used in animals must be a real bone transplanted from the cadaver of another. But it can be very time-consuming and difficult to find a suitable donor, because the variety in size and shape among the animals of a species is far greater than the variety in humans. Dogs, for example, come in a wide variety of breeds and sizes, so their bones correspondingly differ greatly in size and shape. Bones from one breed of dog may not be suitable for use in another, even though the dogs themselves are the same species. Therefore, it can be difficult to obtain the right bone in the right size at the time of the surgery.

In addition, real bones from an animal cadaver or blood bank carries with them the same extensive cleaning, expensive bone cements, and high risks of bacterial or viral infection as found in similar procedures on people. Using natural bone transplantation in animals carries with it all of the problems present in humans in addition to new problems arising from the greater diversity present within animal species.

The use of either artificial prostheses designed for humans or natural bone transplants obtained from cadavers present a number of problems for the animal, the pet owner, and the veterinarian. What is needed is an artificial bone prosthesis specifically designed for use in animals that provides all of the benefits of conventional bone reconstruction therapies without the unnecessary complexities of a human prosthesis or the drawbacks of a natural bone transplant. In addition, it must be easy to manufacture and implant into the animal so that it becomes an inexpensive alternative to conventional artificial bone prostheses.

Related art describes various orthopedic and bone prostheses for use in humans, but none disclose the advantages when used in animals of the present invention.

U.S. Pat. No. 4,040,129 issued on Aug. 9, 1977 to Steinemann et al., describes a bone prosthesis for dental therapeutics with a special chemical composition designed to resist corrosion over a long period of time after implantation into the patient. To accomplish this, the composition of the prosthesis includes very specific concentrations of certain metals and alloys. The specific composition of the alloy distinguishes it over the present invention.

U.S. Pat. No. 4,746,532 issued on May 24, 1988 to Suzuki et al., describes a metallic bone prosthesis that has been coated with a special ceramic material that protects the underlying metal core of the prosthesis from corrosion over the long term. In addition, the fine pores of the ceramic material allow existing bone and surrounding tissue to grow into the prosthesis. This ceramic coating and affinity to bone ingrowth distinguishes this device from the present invention which does not provide for bone ingrowth.

U.S. Pat. No. 4,932,974 issued on Jun. 12, 1990 to Pappas et al., describes bone plates or prostheses that have a microcrystalline grain structure that increases the flexibility of the prosthesis as well as the load on the surrounding bone to facilitate bone healing. This is distinguishable from the present invention which is designed to be rigid.

U.S. Pat. No. 5,281,226 issued on Jan. 25, 1994 to Bavydov et al., describes a bone prosthesis comprising a central rod designed to extend into the medullar canals of adjacent bone fragments and a tubular element fittable on the rod to replace the missing portion of bone. The central rod and tubular element distinguish this device from the present invention, which is designed to be solid and physically separate from the surrounding bone.

U.S. Pat. No. 5,509,933 issued on Apr. 23, 1996 to Davidson et al., describes a medical prosthesis composed of a unique alloy of titanium that has an elasticity close to that of real bone. This is in contrast to the present invention which is designed to be stronger and more rigid than real bone.

U.S. Pat. No. 6,111,164 issued on Aug. 29, 2000 to Rainey et al., describes a bone graft insert which includes a dowel portion made essentially of cortical bone, which distinguishes it from the present invention which is composed entirely of an artificial or synthetic material.

U.S. Pat. No. 6,127,596 issued on Oct. 3, 2000 to Brown et al., describes an orthopedic prosthesis with a ridged and textured surface designed to connect to the surrounding soft tissue of the patient after implantation. This surface distinguishes this device from the present invention which has a substantially smooth surface and which does not require the attachment of soft tissue from the patient.

None of the above inventions and patents, taken either singly or in combination, is seen to describe the instant invention as claimed.

### SUMMARY OF THE INVENTION

The primary difference between the bone reconstruction surgery used in animals and the similar surgery used in humans is that the life span of the animal is greatly reduced. Consequently, unlike the bone prostheses used in humans, bone prostheses used in reconstructive surgery in animals do not have to accommodate tissue or bone ingrowth. Because of the nature of bone cancer and the typical age of affected animals, the animal receiving the prosthesis is not expected to survive long enough for implant failure to occur from repeated cyclic stress.

The present invention takes advantage of this shorter post surgery life span in animals to provide an artificial bone prosthesis with a simple design that is radically different from that used in humans. Specifically, the prosthesis of the present invention uses readily available and easily obtainable materials rather than sophisticated alloys with unique structural characteristics. In addition, the shape of the present invention has a unique simple design that can be used without alteration on a variety of patients rather than having to be custom built to the specifications of a particular patient. The prosthesis of the present invention provides an alternative to complicated, customized human prostheses which have all the benefits of a conventional artificial prosthesis but none of the drawbacks.

The present invention also has a number of benefits over conventional bone transplants as well. The prosthesis of the present invention can be made of a common, readily available artificial or synthetic material that is stronger than bone, such as a prosthesis grade stainless steel. Furthermore, the prosthesis of the present invention requires no preoperative cleaning besides routine sterilization, or preparation and no harvest of bone material from animal cadavers or from bone banks. Because the invention is made of inert artificial materials, it is less likely to promote bacterial or viral infection. Finally, the present invention does not require expensive bone cement in order to join the prosthesis to the surrounding bone, which also promotes infection.

Accordingly, it is a principal object of the invention to provide an artificial bone prosthesis for replacing the missing portion of animal bone that reduces the risk of infection and transmission of disease during implantation.

It is another object of the invention to provide bone prostheses with a simple design that do not require the ingrowth of the surrounding bones.

It is a further object of the invention to provide bone prostheses that can be obtained without cadaver harvest or without resorting to a bone bank.

It is a further object of the invention to provide a bone prosthesis that requires much less preparation before implantation than traditional transplanted allografts.

Still another object of the invention is to provide a bone prosthesis that is stronger and more durable than real bone.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is a perspective view of a stainless steel bone-shaped prosthesis for animals attached to a plate according to the present invention.

FIG. 2 is a rear exploded view of the prosthesis and plate of FIG. 1.

FIG. 3 is an environmental view of the prosthesis device in use in the limb of an animal according to the preferred embodiment.

Similar reference characters denote corresponding features consistently throughout the attached drawings.

### DETAILED DESCRIPTION OF THE PREFERRED EMBODIMENT

The present invention is directed to a stainless steel bone prosthesis for use in veterinary bone replacement surgery.

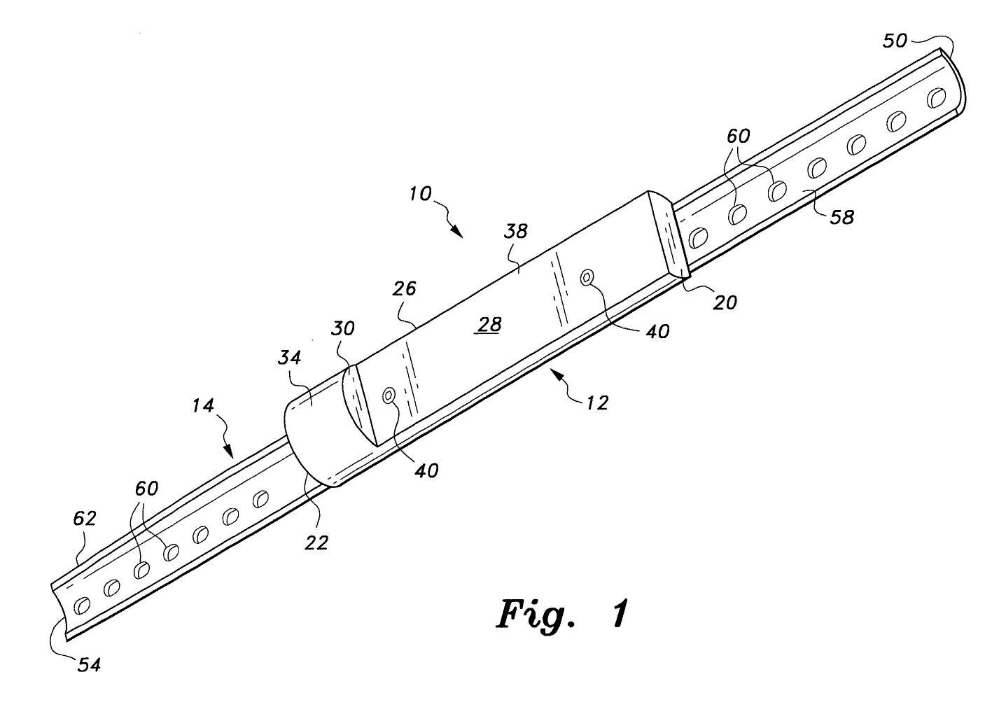

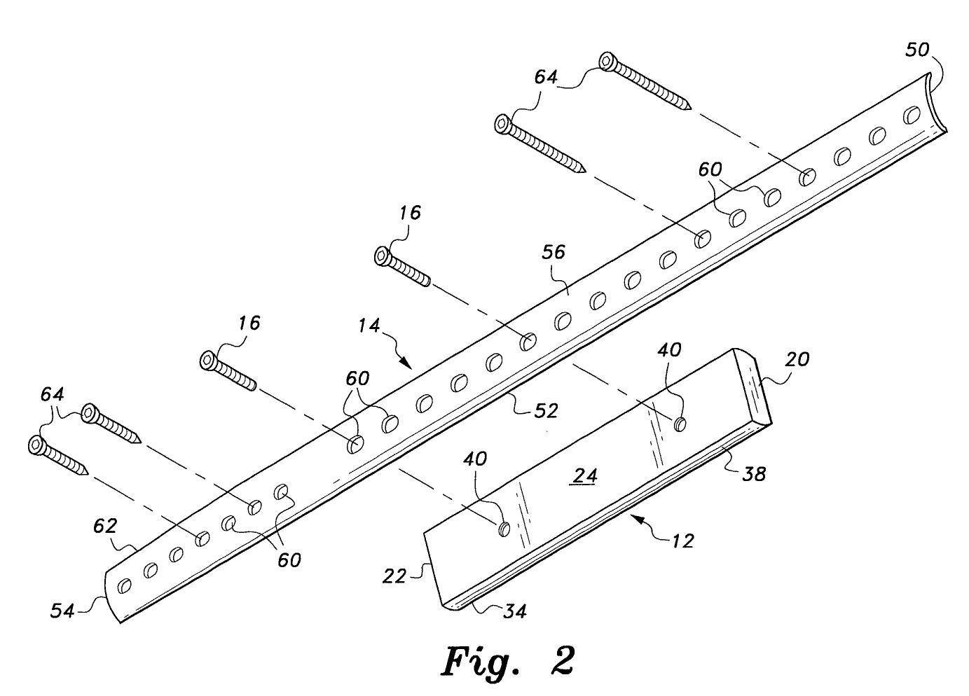

Referring to FIGS. 1 and 2, there are shown a front perspective view and a rear exploded view, respectively, of the prosthesis system of the present invention. Prosthesis system 10 includes prosthesis member 12 attached to plate 14 by machine screws 16. Prosthesis member 12 is generally cylindrical in shape, having an upper shaft abutment end 20 and a lower joint abutment end 22. Prosthesis member 12 has a rear flat 24 extending along the full length thereof and parallel to the vertical axis thereof for mounting to plate 14. Prosthesis member 12 has a front face 26 opposite rear flat 24 having flat 28 extending parallel to rear flat 24 and extending downward from upper shaft abutment end 20 to a notch 30 spaced upward from lower joint abutment end 22 defining member joint abutment portion 34. The front flat 26 extending upward from notch 30 defines member attachment portion 38, said member plate attachment portion 38 having at least two tapped bores 40 spaced therealong for receiving machine screws 42. Abutment end 22 of member joint abutment portion 34 is preferably flat and square to the vertical axis of generally cylindrical prosthesis member 12.

Referring to FIG. 2, plate 14 has an upper end 50 and a lower end 54 and is elongated and substantially planar or semi-cylindrical in form, having an outer convex face 56 and an inner concave face 58(see FIG. 1) extending the length thereof, respectively. Plate 14 has a series of counter sunk screw through bores 60 spaced along its centerline and extending from outer convex face 56 through inner convex face 58. Plate 14 has a central portion 52 about midway between its upper end 50 and lower end 54 where prosthesis member 12 is attached by machine screws 16. Machine screws 16 are inserted through two spaced screw bores 60 and into tapped bores 40 in member plate attachment portion 38, thus, holding plate inner concave face 58 against member rear flat 26.

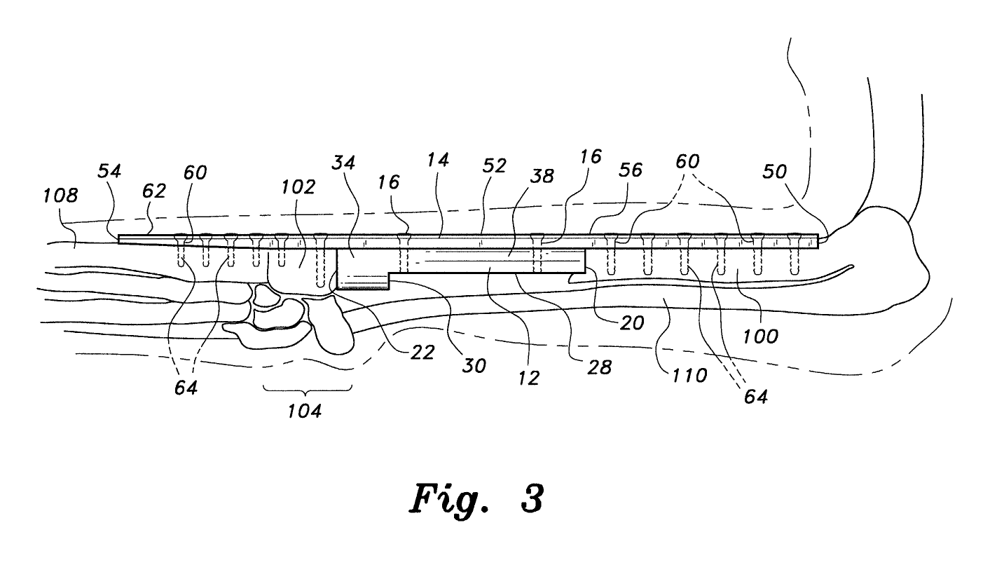

The plate 14 is of such length that when prosthesis member 12 is mounted to plate 14, sufficient screw bores remain exposed both above and below prosthesis member 12 for attachment of the plate to upper and lower bones(see FIG. 3). Plate 14 is tapered in thickness from a point below the mounted prosthesis member 12, the taper extending outward from the inner concave face 58 toward the outer convex face 56 while moving downward from prosthesis member 12 to plate lower end 54, forming lower plate tapered portion 62. Cortical bone screws 64 are inserted through screw bores 60 for attachment to the animal's upper and lower bones(see FIG. 3).

Referring to FIG. 3, the prosthesis system 10 is arranged such that the inner concave face 58 of the upper portion of the plate 12 is mounted by cortical bone screws 64 to the remaining portion of the upper bone 100. Consequently, the lower shaft abutment end 22 lies against the bone 102 and adjacent bones of joint 104, connecting the bone being partially replaced and the lower bone 108 of the limb. The lower portion of plate 14 is connected on its inner concave side to bone 102 by cortical bone screws 64, and the lower tapered portion 62 is likewise attached by cortical bone screws 64 to lower limb bone 108. As shown, the prosthesis 12 is mounted to bone 100 such that front face 26 is facing the coextending bone 110 of the portion of the limb being repaired.

In the preferred embodiment, as illustrated in FIG. 3 and described above, the prosthesis member is used to replace the lower portion of the radius bone of the leg of an animal. In this application, the shaft abutment end lies against the remaining portion of the radius, and the joint abutment end lies against the metacarpals. The joint contact portion is of sufficient length and diameter to make significant surface contact with the carpal bones The front flat of the prosthesis faces the ulna of the limb being repaired, while the rear flat lies flush against the plate. The plate runs from the remaining portion of the radius, across the rear flat of the prosthesis, to the radial carpal bone and metacarpals. As with similar conventional bone allograft surgeries on animal legs, when the plate is screwed into place and the surgery completed, the leg of the animal is fused across the carpal joint.

The prosthesis is made of a conventional 316-L low carbon prosthesis grade stainless steel. The bones are joined to the plate using conventional cortical screws, while the prosthesis is joined using special fine thread screws which fit snugly into the pre drilled holes of the prosthesis.

It is to be understood that the present invention is not limited to the sole embodiment described above, but encompasses any and all embodiments within the scope of the following claims.

I claim:

1. An artificial bone prosthesis system for animals comprising:
an elongated, generally cylindrical, vertically oriented member having an upper shaft abutment end and a lower joint abutment end;
said generally cylindrical member defining a lengthwise vertical axis and having a substantially planar rear flat parallel to said vertical axis, and a front face opposite said rear flat; said member having a plate attachment portion extending downward from said upper shaft abutment end and a member joint abutment end portion extending downward between said plate attachment portion and said lower joint abutment end;
a semi-cylindrical, elongated plate having an upper end and a lower end and a central portion and having a concave inner face and a convex outer face, said plate having a length greater than the length of said prosthesis member, said plate defining at plurality of through bores along the substantial length thereof; and
means for attaching said inner side of said central portion of said plate to said rear flat of said prosthesis member so as to extend parallel therewith;
whereby said plate is mounted to a remaining shaft of an upper bone of a limb such that said member upper shaft abutment end abuts said upper bone; and
whereby said lower joint abutment end abuts a joint; and
whereby said plate extends beyond said joint and is mounted to a lower bone of the limb.

2. The bone prosthesis system of claim 1, wherein said member plate attachment portion defines at least one screw receiving bore extending perpendicularly into said prosthesis member, and said mounting means comprises at least one screw extending from the outer side of said plate, through a corresponding said plate screw bore and into a corresponding said screw receiving bore of said member plate attachment portion.

3. The bone prosthesis system of claim 2, wherein said member plate attachment portion defines two vertically spaced, diametrically directed screw receiving bores and said mounting means comprises two finely threaded machine screws extending therein.

4. The bone prosthesis system of claim 1, wherein said prosthesis member front face has a substantially planar front flat extending downward from said upper shaft abutment end along the length of said plate attachment portion and substantially parallel with said rear flat, said front face defining a notch at the lower end of said attachment portion.

5. The bone prosthesis system of claim 4, wherein said notch extends radially outward, perpendicular to said front flat and extending to the periphery of said front face of said prosthesis member.

6. The bone prosthesis system of claim 5, wherein said notch defines the upper end of said joint abutment portion.

7. The bone prosthesis system of claim 1, wherein said plate has a lower portion between said central portion and said lower end, said lower portion forming a lower tapered portion tapered downwardly from said inner side of said plate toward said outer side of said plate and extending to said plate lower end.

8. The bone prosthesis system of claim 7, further comprising a plurality of cortical bone screws for mounting said plate between said upper end and said central portion to the upper bone shaft and for mounting said lower tapered portion of said plate to said lower bone.

9. The bone prosthesis system of claim 8, wherein said through bores are countersunk within said outer surface of said plate for receiving said bone screws.

10. The bone prosthesis system of claim 1, wherein said mounting plate is substantially planar in configuration.

11. The bone prosthesis of claim 1, wherein said prosthesis is made of 316-L low carbon prosthesis grade stainless steel.

12. An artificial bone prosthesis for animals comprising:
an elongated, generally cylindrical, vertically oriented member having an upper shaft abutment end and a lower joint abutment end;
said generally cylindrical member defining a lengthwise vertical axis and having a substantially planar rear flat parallel to said vertical axis, and a front face opposite said rear flat; said member having a plate attachment portion extending downward from said upper shaft abutment end and a member joint abutment and portion extending downward between said plate attachment portion and said lower joint abutment end.

13. The bone prosthesis of claim 12, wherein said prosthesis member front face has a substantially planar front flat extending downward from said upper shaft abutment end along the length of said plate attachment portion and substantially parallel with said rear flat, said front face defining a notch at the lower end of said attachment portion.

14. The bone prosthesis of claim 13, wherein said notch extends radially outward, perpendicular to said front flat and extending to the periphery of said front face of said prosthesis member.

15. The bone prosthesis of claim 14, wherein said notch defines the upper end of said joint abutment portion.

16. The bone prosthesis of claim 12, wherein said prosthesis is made of 316-L low carbon prosthesis grade stainless steel.
